(19) United States
(12) Patent Application Publication
(10) Pub. No.: US 2025/0367508 A1
(43) Pub. Date: Dec. 4, 2025

(54) SAFETY MECHANISM FOR OPERATING AN EXERCISE EQUIPMENT

(71) Applicant: iFIT Inc., Logan, UT (US)

(72) Inventor: Chris NASCIMENTO, Millville, UT (US)

(21) Appl. No.: 19/222,794

(22) Filed: May 29, 2025

Related U.S. Application Data
(60) Provisional application No. 63/654,638, filed on May 31, 2024.

(51) Int. Cl. A63B 24/00; A63B 22/02; A63B 71/00

(52) U.S. Cl. CPC A63B 24/0087; A63B 22/0235; A63B 71/0054; A63B 2071/0072; A63B 2071/0081

(57) ABSTRACT

An exercise equipment may obtain a motor load of the exercise equipment with selected one or more operating parameters. A motor load may be the amount of energy, the amount of power, the amount of amperage, or the amount of current used to operate the exercise machine with the one or more operating parameters. The exercise equipment may detect a change in the motor load, and in response to detecting the change in the motor load the exercise equipment may slow down the exercise equipment in.

### CROSS REFERENCE TO RELATED APPLICATIONS

The present application for patent claims priority to U.S. Provisional Patent Application No. 63/654,638 by NASCIMENTO, entitled “SAFETY MECHANISM FOR OPERATING AN EXERCISE EQUIPMENT,” filed May 31, 2024, assigned to the assignee hereof, and expressly incorporated by reference in its entirety herein.

### BACKGROUND

Indoor exercise has increased in popularity and accessibility. Many people exercise indoors with the aid of an exercise device. Exercise equipment may be designed to simulate outdoor exercise activities, such as a treadmill to simulate running, a stationary bicycle to simulate cycling, or a rower to simulate rowing. Additionally, or alternatively, exercise equipment may be designed to exercise a certain muscle or muscle group, reduce the impact or force applied to the user, aid in certain types of indoor exercises, perform any other function, and combinations thereof.

Exercise equipment may use electrical power to operate. For example, a treadmill may use electrical power to operate the treadmill belt that a user walks/jogs/runs on. Exercise machines are typically coupled to a mains power supply (through an electrical outlet, for example). Typical commercial treadmill may use max 120 volts in a max 15 Ampere (amp), using a maximum of 1800 watts of power. A direct current (DC) motor controller controls the current provided to the motor that drives the treadmill belt. The more current provided to the motor, the faster the motor may drive the belt.

### BRIEF SUMMARY

In some embodiments, a method for operating an exercise equipment is provided. The method includes obtaining a motor load of the exercise equipment with selected one or more operating parameters. The method further includes detecting a change in the motor load and slowing down the exercise equipment in response to detecting the change in the motor load.

Additionally, or alternatively, in some embodiments, a method for operating an exercise equipment is provided. The method includes operating the exercise equipment with a first configuration. The method further includes obtaining an obtained motor load of the exercise equipment at the first configuration. The method further includes comparing the obtained motor load to an estimated motor load associated with the first configuration. The method further includes performing (e.g., taking) a safety action when the obtained motor load is different than the estimated motor load.

Additionally, or alternatively, in some embodiments, a treadmill configured to prevent injuries is provided. The treadmill includes a motor configured to drive a tread belt of the treadmill. The treadmill further includes a sensor configured to measure a detected motor load. The treadmill further includes a processor and memory, the memory including instructions that cause the processor to (i) operate the treadmill with a first configuration, (ii) receive the detected motor load of the treadmill from the sensor, (iii) compare the detected motor load to an estimated motor load associated with the first configuration, and (iv) perform (e.g., take) a safety action when the detected motor load is different than the estimated motor load.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a representation of an exercise equipment, according to at least one embodiment of the present disclosure.

FIG. 2 illustrates an example of creating a motor load profile, in accordance with at least one or more embodiments.

FIG. 3 illustrates an example of a motor load profile with average current for plurality of users, in accordance with at least one or more embodiments.

FIG. 4 illustrates an example of a motor load profile with minimum, maximum, and average current for a single user based on a speed of a tread belt, in accordance with at least one or more embodiments.

FIG. 5 illustrates an example of a motor load profile with a motor load pattern based on time, in accordance with at least one or more embodiments.

FIG. 6 illustrates an example of detecting a change in the motor load, in accordance with at least one or more embodiments.

FIG. 7 illustrates an example of detecting a change in the motor load, in accordance with at least one or more embodiments.

FIG. 8 illustrates an example of using estimated point of contact information to determine that a user has lost balance, has experienced a moment of unsteadiness, has tripped, or otherwise lost control of him or herself while using an exercise equipment.

FIG. 9 illustrates an example of using estimated point of contact information to determine that a user has lost balance, has experienced a moment of unsteadiness, has tripped, or otherwise lost control of him or herself while using an exercise equipment.

FIG. 10 illustrates a series of acts for operating an exercise equipment, in accordance with one or more embodiments.

FIG. 11 illustrates a series of acts for operating an exercise equipment, in accordance with one or more embodiments.

FIG. 12 illustrates a series of acts for operating a treadmill, in accordance with one or more embodiments.

FIG. 13 illustrates certain components that may be included within a computer system.

### DETAILED DESCRIPTION

This disclosure generally relates to devices, systems, and methods for operating an exercise device. A typical exercise equipment includes one or more input devices that a user may use to change one or more operating parameters of the exercise device. The input device may change one or more operating parameters of the exercise equipment. For example, the input device may change a belt speed of a tread belt on a treadmill, an incline percentage, a resistance level, any other operating parameter, and combinations thereof. During operation of an exercise device, the user may experience moments of unsteadiness, trip, lose balance, or otherwise lose control of him or herself. This may result in injury to the user. A conventional treadmill includes a ‘dead man's switch’ that serves as an emergency stop mechanism. A switch integrated on the treadmill includes a removable clip placed on top of the switch, and a string attaching the removable clip to a user. When the user moves away from the exercising position, the string tightens up and displaces the clip from the top of the switch, and hence activating the switch to perform an emergency stop. The user may not utilize this feature for various reasons and/or the dead man's switch may not work as intended. For example, when running on a treadmill the string attaching the clip to the user may get tangled with the arm movements of the user or otherwise be inconvenient for the user to use, in a way that might trigger an emergency stop when it is not required.

The features and functionalities described herein provide a number of advantages and benefits over conventional approaches and systems. For example, the systems described herein provide features and functionality related to auto-slowdown of a treadmill in response to detecting a change in a motor load of the treadmill. It will be appreciated that the advantages and benefits discussed herein are provided by way of example and are not intended to be an exhaustive list of all possible advantages and benefits of implementations of auto-slowdown functionality and methods described herein.

In some embodiments, a safety action may be performed in response to detecting that an obtained motor load is different from an expected motor load when an exercise equipment is operated with a first configuration. For example, the obtained motor load may be higher or lower than the expected motor load when operated with the first configuration. In some embodiments, the exercise equipment may automatically adjust, without any user input, one or more of the operating parameters. For example, the exercise equipment may adjust one or more of the operating parameters to a neutral operating level. The neutral operating level may be a safe operating level. For example, the neutral operating level may be a stopped tread belt, a slow down to stop a tread belt over a period of time, a low tread belt speed, a low flywheel resistance, a high flywheel resistance that resists rotation of a drivetrain, an interlock on a flywheel or drivetrain to prevent rotation of the drive train, a low incline, zero incline, any other neutral operating level, and combinations thereof. Adjusting the operating parameters to the neutral operating level may help to reduce or prevent injury to the user when he or she trips or otherwise loses balance.

In some embodiments, an action may be performed in response to detecting that an obtained motor load pattern is different from a predictive motor load profile when an exercise equipment is operated with a first configuration. For example, there may be a change in at least one or more of a rhythm of steps, a force applied by each step, or a point of contact made by each step to a tread deck, which may indicate that the user has lost balance, has experienced a moment of unsteadiness, has tripped, or otherwise lost control of him or herself. In response to detecting that the obtained motor load pattern is different from the predictive motor load profile, the exercise equipment may automatically adjust, without any user input, one or more of the operating parameters. For example, the exercise equipment may adjust one or more of the operating parameters to a neutral operating level. The neutral operating level may be a safe operating level. For example, the neutral operating level may be a stopped tread belt, a slow down to stop a tread belt over a period of time, a low tread belt speed, a low flywheel resistance, a high flywheel resistance that resists rotation of a drivetrain, an interlock on a flywheel or drivetrain to prevent rotation of the drive train, a low incline, zero incline, any other neutral operating level, and combinations thereof. Adjusting the operating parameters to the neutral operating level may help to reduce or prevent injury to the user when he or she trips or otherwise loses balance.

In some embodiments, a diagnostic information on a state of health of a treadmill may be provided based on comparing an obtained motor load patter to a predictive motor load profile. For example, if the obtained motor load used for operating a treadmill has increased from the predictive motor load profile, the diagnostic information may suggest one or more maintenance procedures to prevent any further damage and to help extend the service life of the treadmill.

As illustrated in the foregoing discussion, the present disclosure utilizes a variety of terms to described features and advantages of one or more embodiments of motor load detection and methods for facilitating auto-slowdown of an exercise equipment. Additional detail will now be provided regarding the meaning of some of these terms. Further terms will also be discussed in detail in connection with one or more embodiments and specific examples below.

In one or more embodiments described herein, an ‘emergency stop’ is rapid full stop to any movement taking place in the exercise equipment. For example, in treadmill a rapid full stop may mean that all current provided to a motor is prevented. In another example, a rapid full stop for a treadmill may mean that the direction of rotation operated by a motor is reversed or stopped, so as to provide a rapid stop on the movement of the tread belt. In some embodiments, an emergency stop includes a mechanical interference with the operation of one or more movable elements of the treadmill, such as the front pulley, the rear pulley, the tread belt, the belt motor, any other movable element, and combinations thereof.

In one or more embodiments described herein, a ‘slow down’ is a method where the movement of a treadmill is gradually slowed down to a full stop over a period of time. Where an ‘emergency stop’ attempts to stop the movement as quickly as possible, a ‘slow down’ provides a smooth and controlled stopping over a period of time, such as two seconds, three seconds, four seconds, five seconds, six seconds, or over six seconds. For example, a ‘slow down’ may be performed by gradually reducing a power provided to a motor. In another example, an ‘emergency stop’ may be performed by disabling power provided to a motor.

In one or more embodiments, a ‘motor load’ refers to the amount of energy, the amount of power, the amount of amperage, or the amount of current used to move a tread belt. Several different things may affect how much energy, power, amperage, or current is needed to move the tread belt. For example, the speed of the tread belt moving, the incline of the tread deck, the weight of the user on the treadmill, the pattern of force applied by an individual to the treadmill, and wear and tear of various different exercise device modules. A motor load may be obtained by observing or measuring one or more values, such as, a power, an amperage, a current, a resistance, a time, a weight, or a combination thereof.

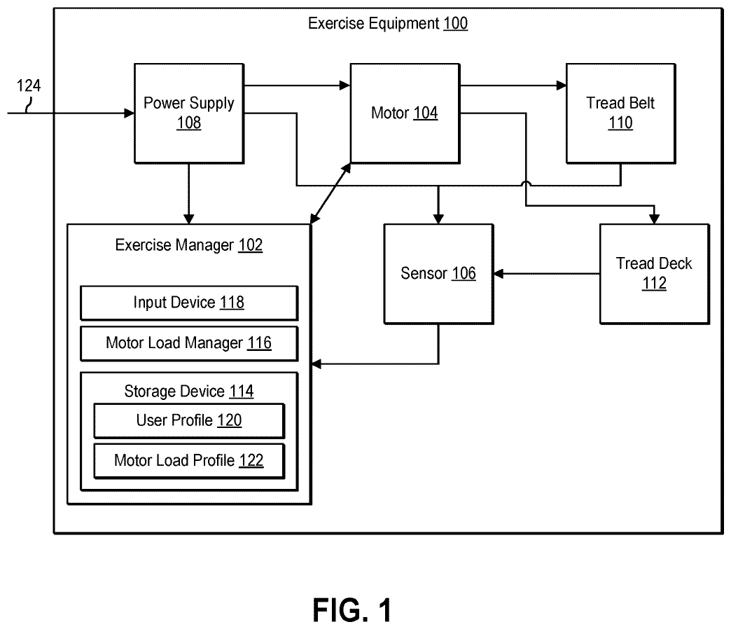

FIG. 1 is a representation of an exercise equipment 100 including a power supply 108, an exercise manager 102, a motor 104, a sensor 106, a tread belt 110, and a tread deck 112, according to at least one embodiment of the present disclosure. In some embodiments, the exercise equipment 100 is configured to receive input power 124, such a regular AC power provided by a household outlet.

In accordance with at least one embodiment of the present disclosure, the exercise equipment 100 may include any type of exercise equipment. For example, the exercise equipment 100 may include a treadmill, an elliptical device, a stationary bicycle, a rower, a cable extension device, any other exercise equipment, and combinations thereof. The exercise equipment 100 may include one or more movable members. For example, a treadmill may include a motor 104 connected to one or both of a front pulley and a rear pulley. A tread belt 110 may be extended between the front pulley and the rear pulley, and rotation of the front pulley and/or the rear pulley may rotate the tread belt 110. The treadmill may include a tread deck 112, and a motor 104 may change an incline of the tread deck 112. In some embodiments, a separate motor may operate the tread belt 110 and the tread deck 112. The exercise equipment 100 may include a flywheel and a movable device to rotate the flywheel, with the flywheel providing resistance to rotation. For example, an elliptical device may include pedals connected to the flywheel such that, when depressed, the pedals may cause the flywheel to rotate, while the flywheel provides resistance to depression of the pedals. In some examples, a stationary bicycle may include pedals and a drivetrain connected to the flywheel and rotation of the pedals may rotate the flywheel. In some examples, a rower and/or a cable extension device may include a cable connected to the flywheel, and extension of the cable may cause the flywheel to rotate.

The exercise equipment 100 may include one or more exercise equipment settings that adjust an operating parameter value of the exercise equipment. The exercise equipment settings may be adjusted, for example, by an input device 118. The exercise equipment settings may include any setting of the exercise equipment 100. For example, the exercise equipment settings may include a tread belt speed, a flywheel resistance, an incline, a decline, a blower fan setting, any other exercise equipment setting, and combinations thereof. In some examples, the exercise equipment 100 may include one or more operating parameters. For example, a thread belt speed may be an exercise equipment setting, and a speed of eight miles per hour may be an operating parameter for this exercise equipment setting. The operating parameters may include one or more of the exercise equipment settings.

In some embodiments, an exercise manager 102 provides one or more input devices 118 a user may use to adjust the exercise equipment settings and/or operating parameters. In some embodiments, the exercise equipment 100 may include multiple input devices 118. For example, the exercise equipment 100 may include a first input device 118 (e.g., a belt input device) for the belt speed of the tread belt 110 and a second input device 118 (e.g., a lift input device) for the incline of the tread deck 112. In some examples, the exercise equipment 100 may include any number of input devices 118 to change any number of operating parameters. In some embodiments, the input device 118 may include a resistance system that may resist movement of the input device 118. The resistance system may include any type of resistance system, such as a spring, a friction fit, a motor, or other resistance. The resistance system may be used to provide the user a tangible sense of movement when moving the input device 118. In some embodiments, the input device 118 may include a return mechanism. The return mechanism may include a spring or compliant material that may return the input device 118 to a neutral position. The user may overpower the return mechanism to adjust the input device 118.

In some embodiments, the exercise equipment 100 includes one or more sensors 106. In some embodiments, the sensor 106 is configured to detect a motor load. For example, the sensor 106 may be a power consumption sensor, an amperage meter, a current meter, a weight sensor, or a combination thereof. The sensor 106 is configured to detect one or more motor load values, such as, a power, an amperage, a current, a weight, a time, or a combination thereof. The sensor 106 may use power provided by the power supply 108 to operate the sensor, or it may have its own power supply. The sensor 106 is configured to provide the one or more detected values to the exercise manager 102. In some embodiments, a motor load is the load used to drive the tread belt 110 of the exercise equipment 100.

In some embodiments, the one or more sensors 106 is configured to detect a point of contact made by each step on the tread deck when an exercise equipment is operated with a first configuration. For example, the one or more sensors 106 may be an optical sensor, electro-mechanical sensors, pneumatic sensors, capacitive sensors, inductive sensors, magnetic sensors, or ultrasonic sensors. These sensors 106 are configured to provide a location and/or the size of area within the tread deck on where a step (e.g., impact) is recorded on. For example, if a location of a step is detected near the sides of the tread deck, or if the size of the step (e.g., impact) is bigger than the estimated size of a feet, it may indicate that the user has lost balance, has experienced a moment of unsteadiness, has tripped, or otherwise lost control of him or herself. In response to detecting that the obtained point of contact is different from the predictive point of contact profile, the exercise equipment may automatically adjust, without any user input, one or more of the operating parameters. For example, the exercise equipment may adjust one or more of the operating parameters to a neutral operating level.

In some embodiments, the exercise manager 102 further includes a storage device 114. A storage device 114 may store user profile 120 that includes one or more user profile parameters, for example, a user's personal information (e.g., age, weight, height, sex, etc.), and the user's preferences on exercise programs (tensity, length, speed, incline, program, etc.).

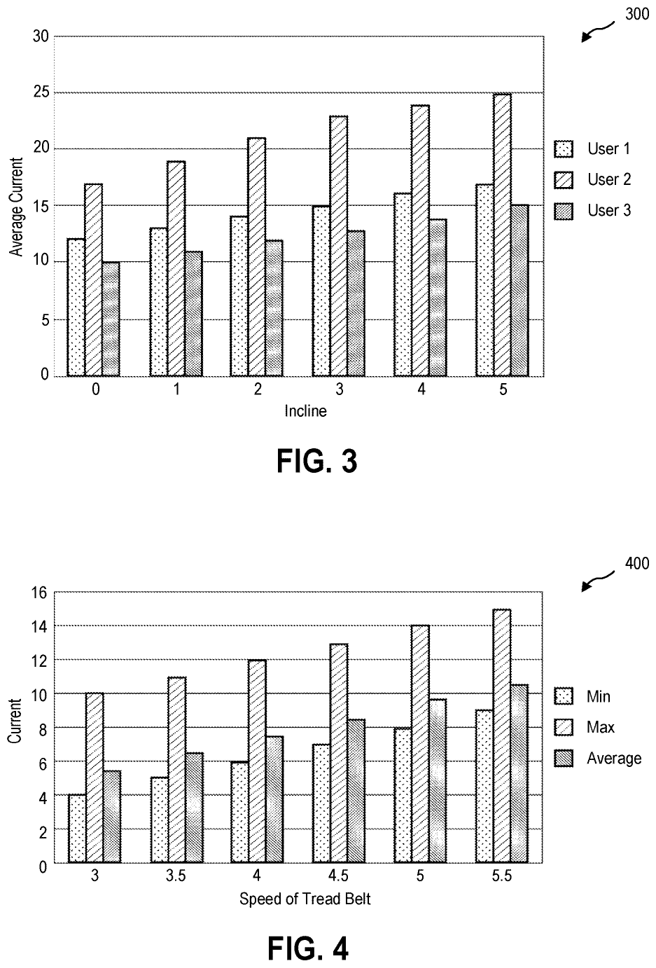

In some embodiments, the exercise manager 102 includes a motor load manager 116 configured to create a motor load profile 122 for a user. For example, the motor load manager 116 may analyze the one or more user profile parameters, the one or more exercise equipment settings, the one or more motor load values, the one or more point of contact point information, or a combination thereof to create a motor load profile 122 for the user. For example, a motor load profile may include one or more of an average motor load, a motor load pattern over a period of time, a maximum motor load, a minimum motor load, a point of contact information, or a combination thereof. Examples of a motor load profile are further discussed in connection to FIGS. 3-5. The motor load profile 122 may be stored on the storage device 114. In some embodiments, the motor load profile is stored on an external storage device via a network connection.

In some embodiments, the motor load manager 116 is further configured to compare the motor load profile 122 to an obtained motor load values and/or the obtained point of contact information provided by the sensor 106. For example, by comparing the motor load profile 122 to the obtained motor load values, the exercise manager 102 may detect a change in the motor load. In response to detecting the change in the motor load, the exercise manager 102 may perform (e.g., take) a safety action, such as adjusting operation of the exercise equipment (e.g., initiating an emergency stop, or initiating a slowdown), providing a warning (e.g., providing an audible alert sound and/or providing a warning to a user via a display), or a combination thereof. In some embodiments, providing a warning to a user via a display further includes providing a prompt to confirm identity of the individual using the exercise device. For example, a first user may have a different motor load profile than a second user, and hence the second user may trigger a difference on a motor load if operated with the assumption that it is the first user operating it. In some embodiment, the motor load manager 116 may the utilize point of contact information in determining what type of safety action to perform based on the change in the motor load.

In some embodiments, the motor load profile may be based on one or more exercises performed in the past by a user. In some embodiments, the motor load profile may be based on the ongoing (e.g., current) exercise performed by a user. In some embodiments, the motor load profile may be a combination of the past exercise performances and the current exercise performance.

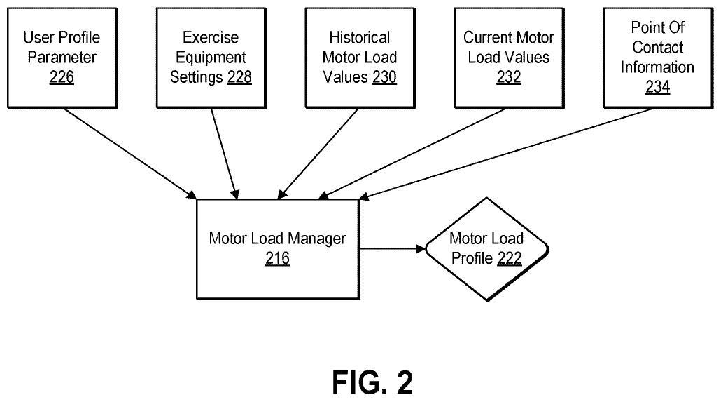

FIG. 2 illustrates an example of creating a motor load profile, in accordance with at least one or more embodiments. The motor load manager 216 may receive one or more user profile parameters 226, one or more exercise equipment settings 228, one or more historical motor load values 230, one or more current motor load values 232, one or more point of contact information 234, or a combination thereof. The motor load manager 216 may utilize one or more of these to create a motor load profile 222 for a user. For example, a motor load profile may include one or more of an average motor load, a motor load pattern over a period of time, a maximum motor load, a minimum motor load, a point of contact pattern, or a combination thereof.

In some embodiments, a motor load profile 222 includes an average estimated motor load required to operate the exercise machine for a first user with a first set of operating parameters. For example, the motor load manager 216 may use a first user's weight (e.g., user profile parameters 226), and a first speed of a tread belt to estimate an average motor load for the first user using the exercise equipment with the first speed. The motor load manager 216 may then perform similar analysis with a second speed, third speed, etc., or to a second user, third user, etc.

In some embodiments, a motor load profile may include an estimated minimum (min) and/or maximum (max) motor load required to operate the exercise machine for a first user with a first set of operating parameters. For example, the motor load manager 216 may receive current exercise equipment settings, and current motor load values to estimate min and max motor load required to operate the exercise machine with the current exercise equipment settings.

In some embodiment, the motor load profile 222 includes a motor load pattern over a period of time. For example, motor load manager 216 may use historical motor load values detected by a motor load sensor (such as the sensor 106 of FIG. 1) and stored by a storage device (such as the storage device 114 of FIG. 1) to create a motor load pattern over a period of time. In some embodiments, a motor load pattern may show a change in motor load over a period of time. For example, in treadmill, the motor load value may be higher each time a feet touches the treadmill, compared to times when no feet are touching the treadmill such as when a user is running on a treadmill. In another example, the impact of a feet touching a treadmill when a user is walking may be lower than the impact of a feet touching a treadmill when the user is running. For example, with an exercise bike, the rhythm of pedaling may be different based on the exercise equipment settings used, the fitness of the user, and the strength of both feet (e.g., imbalance on muscle strength and/or injuries on a leg may affect how much workload each pedaling requires). Similarly with a flywheel, the exercise equipment settings, and the fitness of the user may affect how much workload is required.

In some embodiments, a motor load profile 222 additionally includes a point of contact pattern. For example, one or more sensors, such as the sensor 106 in FIG. 1, may detect a point of contact made by each step on the tread deck when an exercise equipment is operated with a first configuration. For example, the one or more sensors may be an optical sensor, electro-mechanical sensors, pneumatic sensors, capacitive sensors, inductive sensors, magnetic sensors, or ultrasonic sensors. In some embodiments, the sensors are configured to provide a point of contact information 234 to the motor load manager 216. For example, the point of contact information 234 may be a position (e.g., a location) within the tread belt where a step has been detected, and/or the size of area within the tread deck on where a step (e.g., impact) is detected on. Based on the received point of contact information 234 the motor load manager 216 may perform a statistical analysis to determine estimated point of contact, e.g., the estimated position and/or size of the impact on the tread belt which is stored as part of the motor load profile 222. The estimated point of contact may then be used together with the motor load estimation to determine if the user has lost balance, has experienced a moment of unsteadiness, has tripped, or otherwise lost control of him or herself. For example, if a location of a step is detected near the sides of the tread deck, or if the size of the step (e.g., impact) is bigger than the estimated size of a feet, it may indicate that the user has lost balance, has experienced a moment of unsteadiness, has tripped, or otherwise lost control of him or herself. In response to detecting that the obtained point of contact is different from the predictive point of contact profile, the exercise equipment may automatically adjust, without any user input, one or more of the operating parameters. For example, the exercise equipment may adjust one or more of the operating parameters to a neutral operating level.

FIG. 3 illustrates an example of a motor load profile 300 with average current for plurality of users, in accordance with at least one or more embodiments. In FIG. 3, an average current required to operate a treadmill with six different incline parameters for three different users are provided. As can be seen, the average current with User 3 is lower than the average current required for Users 1 and 2. Differences on the average current for different users may be explained by the weight of the users, or the speed of the treadmill. For example, here, User 3 may weigh the least and User 2 may weigh the most of the three. In another example, User 2 may always operate the treadmill on higher speed than Users 1 and 3. For example, in FIG. 3, User 2 may operate the treadmill on 5 mph speed, User 1 may operate the treadmill on 3 mph, and User 3 may operate the treadmill on 2.5 mph during each incline.

FIG. 4 illustrates an example of a motor load profile 400 with minimum, maximum, and average current for a single user based on a speed of a tread belt, in accordance with at least one or more embodiments. As shown in FIG. 4, as the speed of the tread belt increases, so does the minimum, maximum, and average current required to operate the exercise machine increase. In some embodiments, the increase may or may not be linear.

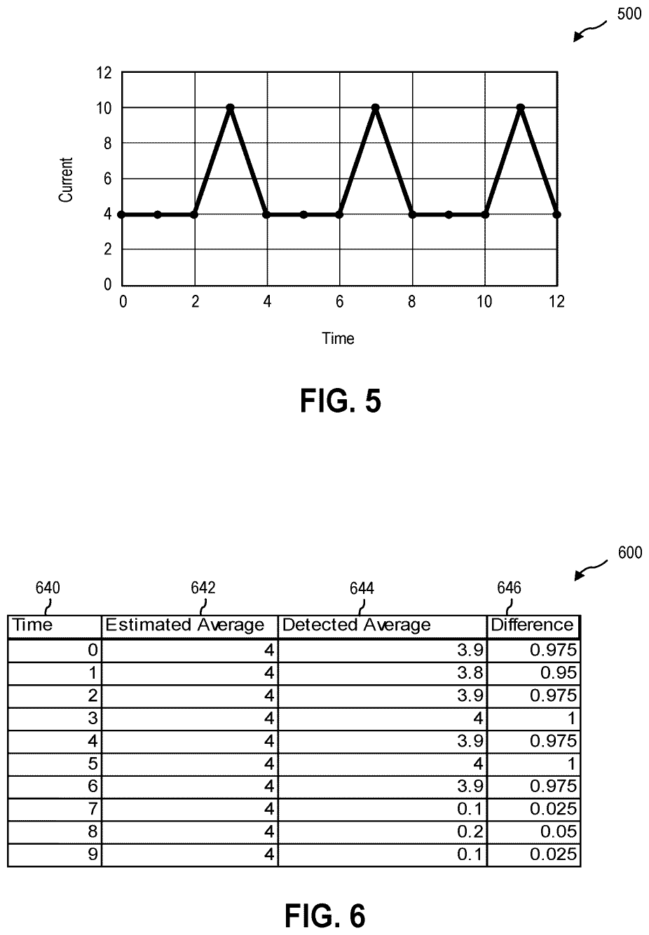

FIG. 5 illustrates an example of a motor load profile 500 with a motor load pattern based on time, in accordance with at least one or more embodiments. For example, the motor load pattern in FIG. 5, may be for a treadmill. As shown in FIG. 5, during t=0 and t=2, the current required to operate a treadmill is 4 amps. At t=3, the current required to operate the treadmill increases to 10 amps. During t=4 and t=6, the current required to operate the treadmill decreases back to 4 amps. At t=7, the current required to operate the treadmill increases again to 10 amps. As can be seen by the motor load profile 500, this same pattern of three consecutive time instances at 4 amps followed by a single time instance at 10 amps continues along the whole time frame shown on the motor load profile 500. It can be concluded that at t=3, t=7, and t=11 is the time frame when a user's feet touches the treadmill, for example, when the user is running and providing heavier weight on the tread belt on those time instances when their feet touches the tread belt.

In some examples, FIG. 5 may represent the motor load for a flywheel. For example, at t=3, t=7, and t=11 is the time frame when the user pulls the flywheel, and the exercise device provides resistance to the pull. It should be noted that even if the motor load value shown in FIGS. 3-5 is a current, any other motor load value could also be used (e.g., a power, an amperage, a current, a resistance, or a combination thereof).

FIG. 6 illustrates an example 600 of detecting a change in the motor load, in accordance with at least one or more embodiments. In the example 600 shown in FIG. 6, over a period of time 640 from t=0 to t=9, the average estimated motor load 642 (e.g., power, amperage, current, resistance, or combination thereof) has been determined to be 4 units. A detected average of motor load 644 has been detected for the same time periods, and the difference 646 between the two has been provided. The closer to 1 the difference 646 is, the more aligned the estimated motor load 642 and the detected motor load 644 are to each other's. As can be seen from FIG. 6, there is not much difference between the two, between t=0 and t=6. However, the exercise equipment has detected significantly decreased motor load values between t=7 and t=9. For example, a significantly decreased motor load values may indicate that the user has fallen off the exercise device or has voluntarily jumped out of it. In some embodiments, a threshold value may be set on how big of a difference is allowed between the estimated average and the detected average to determine that the difference is significant. For example, a difference between 0.9 and 1.1 may be accepted as normal whereas anything below 0.9 or above 1.1 is considered significant. In another example, a difference between 0.8 and 1.2 may be accepted as normal whereas anything below 0.8 or above 1.2 is considered significant.

In response to detecting a significant change on the expected and detected motor load values, the exercise device may perform (e.g., take) an action. For example, an action may include adjusting operation of the treadmill to a neutral configuration. In some embodiments, a time threshold may also be set on how quickly an action is performed based on detecting a significant difference. For example, if there is a significant different on two consecutive time periods, the system may perform a safety action. In another example, if there is three or more consecutive time periods, the system may perform (e.g., take) a safety action. In yet another example, detecting only one significant difference may be enough to initiate a safety action.

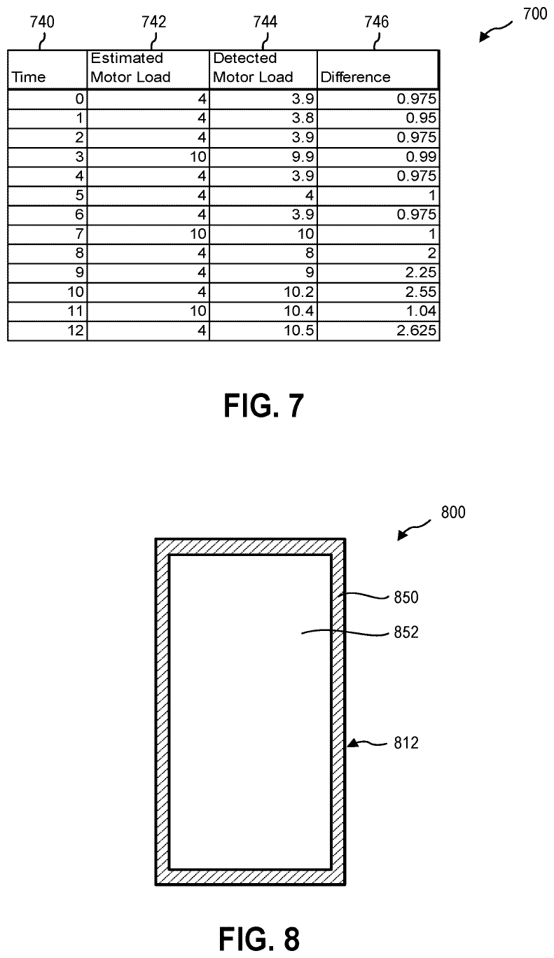

FIG. 7 illustrates an example 700 of detecting a change in the motor load, in accordance with at least one or more embodiments. In the example 700 shown in FIG. 7, the estimated value 742 of motor load is based on the motor load profile created in connection to FIG. 5. The difference 746 between the estimated value 742 and the detected motor load value 744 over time 740 may be non-significant between t=0 and t=7. Between t=8 and t=10, there may be significant difference 746 detected, while at t=11 the difference 746 may not be significant. For example, as the detected motor load value 744 is significantly higher than expected (i.e., when the user's feet are not touching the tread belt) it can be concluded that the user has most probably fallen on to the belt and is not running anymore, as the detected value between t=8 and t=12 stays around the same (high) value.

In some embodiments, a minimum and maximum values for the motor load may also be used to detect if the detected average or the detected value falls outside of the minimum and maximum values, in which case it is determined that there is a significant change in the motor load value.

FIG. 8 illustrates an example 800 of using estimated point of contact information to determine that a user has lost balance, has experienced a moment of unsteadiness, has tripped, or otherwise lost control of him or herself while using an exercise equipment. In some embodiments, a motor load profile, such as the motor load profile 222 of FIG. 2, includes estimation of point of contact on a tread belt 812. For example, the estimation may be based on sensor data collected over time, as previously discussed in connection to FIG. 2, or it could be automatically set. As shown in FIG. 8, a danger zone 850 has been established to cover the boarders of the tread belt 812. Similarly, a safe zone 852 has been provided to cover the rest of the area of the tread belt 812. In some embodiments, when an impact is detected on a danger zone 850 the system may determine that the user has lost balance, has experienced a moment of unsteadiness, has tripped, or otherwise lost control of him or herself. In some embodiments, when an impact is detected on a danger zone 850 and if a difference is detected on a workload, the system may determine that the user has lost balance, has experienced a moment of unsteadiness, has tripped, or otherwise lost control of him or herself.

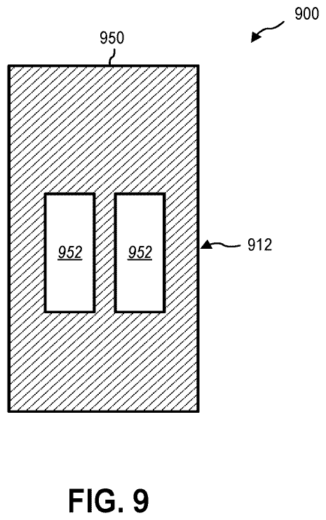

FIG. 9 illustrates an example 900 of using estimated point of contact information to determine that a user has lost balance, has experienced a moment of unsteadiness, has tripped, or otherwise lost control of him or herself while using an exercise equipment. In some embodiments, a motor load profile, such as the motor load profile 222 of FIG. 2, includes estimation of point of contact on a tread belt 912. For example, the estimation may be based on sensor data collected over time, as previously discussed in connection to FIG. 2, or it could be automatically set. As shown in FIG. 9, two separate safe zones 952 has been established based on sensor data collected when the exercise machine is operated with first configuration. In some embodiments, the two separate safe zones 952 may be separately established for each feet. For example, if a left feet is detected on a safe zone that has been established for right feet, and which is outside of the left feet safe zone, it can still be determined to be on a danger zone for the left feet. Similarly, if a right feet is detected outside of right feet safe zone, even if it would be on a left feet safe zone, it will still be determined to be on a danger zone for right feet. For simplicity, the two separate safe zones 952 shown on FIG. 9, are here determined to be safe zones for both feet. A danger zone 950 covers the rest of the area of the tread belt 912. In some embodiments, when an impact is detected on a danger zone 950 the system may determine that the user has lost balanced, has experienced a moment of unsteadiness, has tripped, or otherwise lost control of him or herself. In some embodiments, when an impact is detected on a danger zone 950 and if a difference is detected on a workload, the system may determine that the user has lost balanced, has experienced a moment of unsteadiness, has tripped, or otherwise lost control of him or herself. In response to detecting that the obtained point of contact is different from the predictive point of contact profile, the exercise equipment may automatically adjust, without any user input, one or more of the operating parameters. For example, the exercise equipment may adjust one or more of the operating parameters to a neutral operating level.

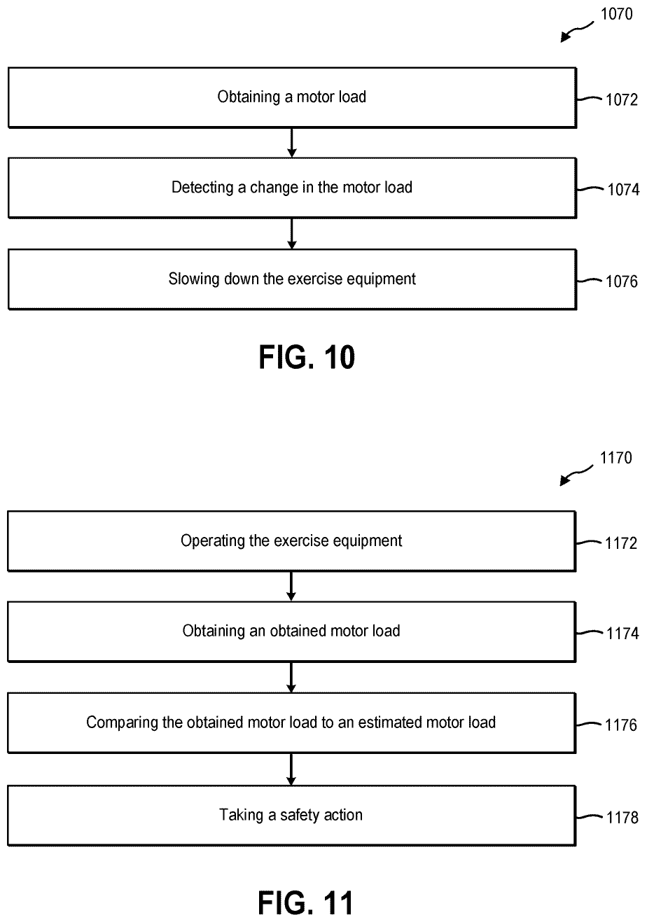

FIG. 10 illustrates a series of acts 1070 for operating an exercise equipment, in accordance with one or more embodiments. While FIG. 10 illustrates acts according to one or more embodiments, alternative embodiments may omit, add to, reorder, and/or modify any of the acts shown in FIG. 10. The acts of FIG. 10 can be performed as part of a method. Alternatively, a system or a device can perform the acts of FIG. 10.

As shown in FIG. 10, the series of acts 1070 may include an act 1072 of obtaining a motor load. For example, the motor load may be the motor load of an exercise equipment with selected one or more operating parameters.

The series of acts 1070 may further include an act 1074 of detecting a change in the motor load. For example, detecting the change in the motor load may include comparing a current used to drive the exercise equipment with a selected operating parameters to an estimated current used to drive the exercise equipment with the selected operating parameters and detecting over 10% difference between the two.

The series of acts 1070 may further include an act 1076 of slowing down the exercise equipment in response to detecting the change in the motor load. For example, slowing down the exercise equipment may be done automatically without user input.

FIG. 11 illustrates a series of acts 1170 for operating an exercise equipment, in accordance with one or more embodiments. While FIG. 11 illustrates acts according to one or more embodiments, alternative embodiments may omit, add to, reorder, and/or modify any of the acts shown in FIG. 11. The acts of FIG. 11 can be performed as part of a method. Alternatively, a system or a device can perform the acts of FIG. 11.

As shown in FIG. 11, the series of acts 1170 may include an act 1172 of operating the exercise equipment. For example, operating the exercise equipment with a first configuration.

The series of acts 1170 may further include an act 1174 of obtaining an obtained motor load. For example, obtaining an obtained motor load of the exercise equipment at the first configuration.

The series of acts 1170 may further include an act 1176 of comparing the obtained motor load to an estimated motor load. For example, comparing the obtained motor load to an estimated motor load associated with the first configuration.

The series of acts 1170 may further include an act 1178 of performing (e.g., taking) a safety action. For example, performing a safety action when the obtained motor load is different than the estimated motor load.

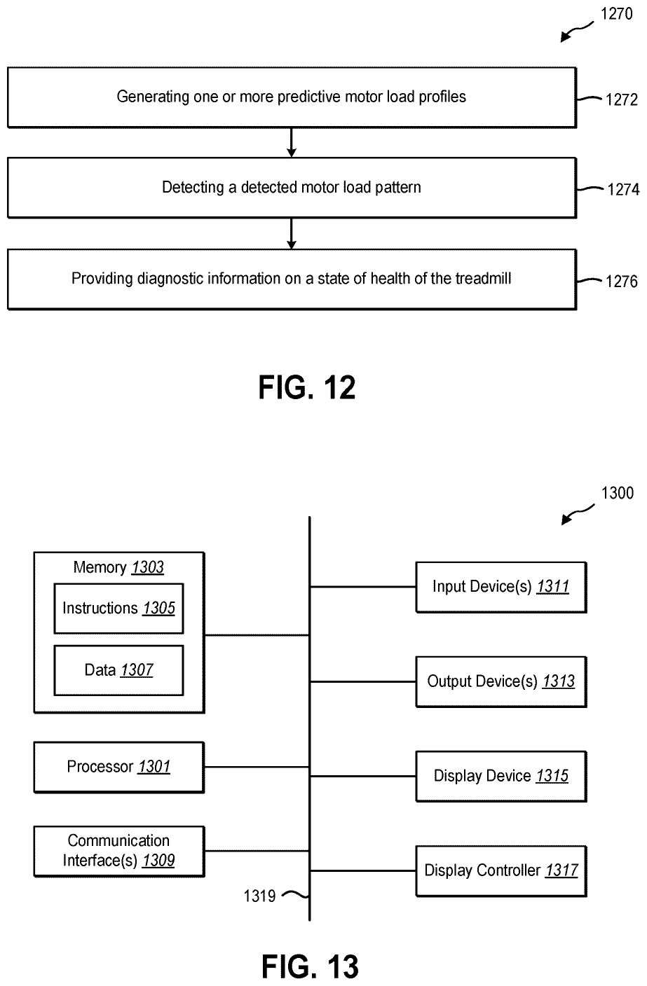

FIG. 12 illustrates a series of acts 1270 for operating a treadmill in accordance with one or more embodiments. While FIG. 12 illustrates acts according to one or more embodiments, alternative embodiments may omit, add to, reorder, and/or modify any of the acts shown in FIG. 12. The acts of FIG. 12 can be performed as part of a method. Alternatively, a system or a device can perform the acts of FIG. 12.

As shown in FIG. 12, the series of acts 1270 may include an act 1272 of generating one or more predictive motor load profiles. For example, generating one or more predictive motor load profiles for an individual user based on historical motor load information of the treadmill at an operating configuration.

The series of acts 1270 may further include an act 1274 of detecting a detected motor load pattern. For example, detecting a detected motor load pattern of the treadmill at the operating configuration for the individual user.

The series of acts 1270 may further include an act 1276 of providing diagnostic information on a state of health of the treadmill. For example, providing diagnostic information on a state of health of the treadmill may be provided based on comparing an obtained motor load patter to a predictive motor load profile. For example, if the obtained motor load used for operating the exercise equipment has increased from the predictive motor load profile, the diagnostic information may suggest one or more maintenance procedures to prevent any further damage and to help extend the service life of the treadmill.

FIG. 13 illustrates certain components that may be included within a computer system 1300. The computer system 1300 may be used to implement the various computing devices, components, and systems described herein. As used herein, a “computing device” refers to electronic components that perform a set of operations based on a set of programmed instructions. Computing devices include groups of electronic components, client devices, server devices, etc.

In various implementations, the computer system 1300 represents one or more of the client devices, server devices, or other computing devices described above. For example, the computer system 1300 may refer to various types of network devices capable of accessing data on a network, a cloud computing system, or another system. For instance, a client device may refer to a mobile device such as a mobile telephone, a smartphone, a personal digital assistant (PDA), a tablet, a laptop, a wearable computing device (e.g., a headset or smartwatch), or exercise device. A client device may also refer to a non-mobile device such as a desktop computer, a server node (e.g., from another cloud computing system), or another non-portable device.

The computer system 1300 includes a processing system including a processor 1301. The processor 1301 may be a general-purpose single- or multi-chip microprocessor (e.g., an Advanced Reduced Instruction Set Computer (RISC) Machine (ARM)), a special-purpose microprocessor (e.g., a digital signal processor (DSP)), a microcontroller, a programmable gate array, etc. The processor 1301 may be referred to as a central processing unit (CPU). Although the processor 1301 shown is just a single processor in the computer system 1300 of FIG. 13, in an alternative configuration, a combination of processors (e.g., an ARM and DSP) could be used.

The computer system 1300 also includes memory 1303 in electronic communication with the processor 1301. The memory 1303 may be any electronic component capable of storing electronic information. For example, the memory 1303 may be embodied as random-access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM), registers, and so forth, including combinations thereof.

The instructions 1305 and the data 1307 may be stored in the memory 1303. The instructions 1305 may be executable by the processor 1301 to implement some or all of the functionality disclosed herein. Executing the instructions 1305 may involve the use of the data 1307 that is stored in the memory 1303. Any of the various examples of modules and components described herein may be implemented, partially or wholly, as instructions 1305 stored in memory 1303 and executed by the processor 1301. Any of the various examples of data described herein may be among the data 1307 that is stored in memory 1303 and used during the execution of the instructions 1305 by the processor 1301.

A computer system 1300 may also include one or more communication interface(s) 1309 for communicating with other electronic devices. The one or more communication interface(s) 1309 may be based on wired communication technology, wireless communication technology, or both. Some examples of the one or more communication interface(s) 1309 include a Universal Serial Bus (USB), an Ethernet adapter, a wireless adapter that operates in accordance with an Institute of Electrical and Electronics Engineers (IEEE) 1002.11 wireless communication protocol, a Bluetooth® wireless communication adapter, and an infrared (IR) communication port.

A computer system 1300 may also include one or more input device(s) 1311 and one or more output device(s) 1313. Some examples of the one or more input device(s) 1311 include a keyboard, mouse, microphone, remote control device, button, joystick, trackball, touchpad, and light pen. Some examples of the one or more output device(s) 1313 include a speaker and a printer. A specific type of output device that is typically included in a computer system 1300 is a display device 1315. The display device 1315 used with implementations disclosed herein may utilize any suitable image projection technology, such as liquid crystal display (LCD), light-emitting diode (LED), gas plasma, electroluminescence, or the like. A display controller 1317 may also be provided, for converting data 1307 stored in the memory 1303 into text, graphics, and/or moving images (as appropriate) shown on the display device 1315.

The various components of the computer system 1300 may be coupled together by one or more buses, which may include a power bus, a control signal bus, a status signal bus, a data bus, etc. For clarity, the various buses are illustrated in FIG. 13 as a bus system 1319.

### INDUSTRIAL APPLICABILITY

This disclosure generally relates to devices, systems, and methods for operating an exercise device. A typical exercise equipment includes one or more input devices that a user may use to change one or more operating parameters of the exercise device. The input device may change one or more operating parameters of the exercise equipment. For example, the input device may change a belt speed of a tread belt on a treadmill, an incline percentage, a resistance level, any other operating parameter, and combinations thereof. During operation of an exercise device, the user may experience moments of unsteadiness, trip, lose balance, or otherwise lose control of him or herself. This may result in injury to the user. A conventional treadmill includes a ‘dead man's switch’ that serves as an emergency stop mechanism. A switch integrated on the treadmill includes a removable clip placed on top of the switch, and a string attaching the removable clip to a user. When the user moves away from the exercising position, the string tightens up and displaces the clip from the top of the switch, and hence activating the switch to perform an emergency stop. The user may not utilize this feature for various reasons and/or the dead man's switch may not work as intended. For example, when running on a treadmill the string attaching the clip to the user may get tangled with the arm movements of the user or otherwise be inconvenient for the user to use, in a way that might trigger an emergency stop when it is not required.

The features and functionalities described herein provide a number of advantages and benefits over conventional approaches and systems. For example, the systems described herein provide features and functionality related to auto-slowdown of a treadmill in response to detecting a change in a motor load of the treadmill. It will be appreciated that the advantages and benefits discussed herein are provided by way of example and are not intended to be an exhaustive list of all possible advantages and benefits of implementations of auto-slowdown functionality and methods described herein.

In some embodiments, a safety action may be performed in response to detecting that an obtained motor load is different from an expected motor load when an exercise equipment is operated with a first configuration. For example, the obtained motor load may be higher or lower than the expected motor load when operated with the first configuration. In some embodiments, the exercise equipment may automatically adjust, without any user input, one or more of the operating parameters. For example, the exercise equipment may adjust one or more of the operating parameters to a neutral operating level. The neutral operating level may be a safe operating level. For example, the neutral operating level may be a stopped tread belt, a slow down to stop a tread belt over a period of time, a low tread belt speed, a low flywheel resistance, a high flywheel resistance that resists rotation of a drivetrain, an interlock on a flywheel or drivetrain to prevent rotation of the drive train, a low incline, zero incline, any other neutral operating level, and combinations thereof. Adjusting the operating parameters to the neutral operating level may help to reduce or prevent injury to the user when he or she trips or otherwise loses balance.

In some embodiments, an action may be performed in response to detecting that an obtained motor load pattern is different from a predictive motor load profile when an exercise equipment is operated with a first configuration. For example, there may be a change in at least one or more of a rhythm of steps, a force applied by each step, or a point of contact made by each step to a tread deck, which may indicate that the user has lost balance, has experienced a moment of unsteadiness, has tripped, or otherwise lost control of him or herself. In response to detecting that the obtained motor load pattern is different from the predictive motor load profile, the exercise equipment may automatically adjust, without any user input, one or more of the operating parameters. For example, the exercise equipment may adjust one or more of the operating parameters to a neutral operating level. The neutral operating level may be a safe operating level. For example, the neutral operating level may be a stopped tread belt, a slow down to stop a tread belt over a period of time, a low tread belt speed, a low flywheel resistance, a high flywheel resistance that resists rotation of a drivetrain, an interlock on a flywheel or drivetrain to prevent rotation of the drive train, a low incline, zero incline, any other neutral operating level, and combinations thereof. Adjusting the operating parameters to the neutral operating level may help to reduce or prevent injury to the user when he or she trips or otherwise loses balance.

In some embodiments, a diagnostic information on a state of health of a treadmill may be provided based on comparing an obtained motor load patter to a predictive motor load profile. For example, if the obtained motor load used for operating a treadmill has increased from the predictive motor load profile, the diagnostic information may suggest one or more maintenance procedures to prevent any further damage and to help extend the service life of the treadmill.

As illustrated in the foregoing discussion, the present disclosure utilizes a variety of terms to described features and advantages of one or more embodiments of motor load detection and methods for facilitating auto-slowdown of an exercise equipment. Additional detail will now be provided regarding the meaning of some of these terms. Further terms will also be discussed in detail in connection with one or more embodiments and specific examples below.

In one or more embodiments described herein, an ‘emergency stop’ is rapid full stop to any movement taking place in the exercise equipment. For example, in treadmill a rapid full stop may mean that all current provided to a motor is prevented. In another example, a rapid full stop for a treadmill may mean that the direction of rotation operated by a motor is reversed or stopped, so as to provide a rapid stop on the movement of the tread belt. In some embodiments, an emergency stop includes a mechanical interference with the operation of one or more movable elements of the treadmill, such as the front pulley, the rear pulley, the tread belt, the belt motor, any other movable element, and combinations thereof.

In one or more embodiments described herein, a ‘slow down’ is a method where the movement of a treadmill is gradually slowed down to a full stop over a period of time. Where an ‘emergency stop’ attempts to stop the movement as quickly as possible, a ‘slow down’ provides a smooth and controlled stopping over a period of time, such as two seconds, three seconds, four seconds, five seconds, six seconds, or over six seconds. For example, a ‘slow down’ may be performed by gradually reducing a power provided to a motor. In another example, an ‘emergency stop’ may be performed by disabling power provided to a motor.

In one or more embodiments, a ‘motor load’ refers to the amount of energy, the amount of power, the amount of amperage, or the amount of current used to move a tread belt. Several different things may affect how much energy, power, amperage, or current is needed to move the tread belt. For example, the speed of the tread belt moving, the incline of the tread deck, the weight of the user on the treadmill, the pattern of force applied by an individual to the treadmill, and wear and tear of various different exercise device modules. A motor load may be obtained by observing or measuring one or more values, such as, a power, an amperage, a current, a resistance, a time, a weight, or a combination thereof.

In some embodiments, an exercise equipment includes a power supply, an exercise manager, a motor, a sensor, a tread belt, and a tread deck, according to at least one embodiment of the present disclosure. In some embodiments, the exercise equipment is configured to receive input power, such a regular AC power provided by a household outlet.

In accordance with at least one embodiment of the present disclosure, the exercise equipment may include any type of exercise equipment. For example, the exercise equipment may include a treadmill, an elliptical device, a stationary bicycle, a rower, a cable extension device, any other exercise equipment, and combinations thereof. The exercise equipment may include one or more movable members. For example, a treadmill may include a motor connected to one or both of a front pulley and a rear pulley. A tread belt may be extended between the front pulley and the rear pulley, and rotation of the front pulley and/or the rear pulley may rotate the tread belt. The treadmill may include a tread deck, and a motor may change an incline of the tread deck. In some embodiments, a separate motor may operate the tread belt and the tread deck. One or more exercise equipment may include a flywheel and a movable device to rotate the flywheel, with the flywheel providing resistance to rotation. For example, an elliptical device may include pedals connected to the flywheel such that, when depressed, the pedals may cause the flywheel to rotate, while the flywheel provides resistance to depression of the pedals. In some examples, a stationary bicycle may include pedals and a drivetrain connected to the flywheel and rotation of the pedals may rotate the flywheel. In some examples, a rower and/or a cable extension device may include a cable connected to the flywheel, and extension of the cable may cause the flywheel to rotate.

The exercise equipment may include one or more exercise equipment settings that adjust an operating parameter value of the exercise equipment. The exercise equipment settings may be adjusted, for example, by an input device. The exercise equipment settings may include any setting of the exercise equipment. For example, the exercise equipment settings may include a tread belt speed, a flywheel resistance, an incline, a decline, a blower fan setting, any other exercise equipment setting, and combinations thereof. In some examples, the exercise equipment may include one or more operating parameters. For example, a thread belt speed may be an exercise equipment setting, and a speed of eight miles per hour may be an operating parameter for this exercise equipment setting. The operating parameters may include one or more of the exercise equipment settings.

In some embodiments, an exercise manager provides one or more input devices a user may use to adjust the exercise equipment settings and/or operating parameters. In some embodiments, the exercise equipment may include multiple input devices. For example, the exercise equipment may include a first input device (e.g., a belt input device) for the belt speed of the tread belt and a second input device (e.g., a lift input device) for the incline of the tread deck. In some examples, the exercise equipment may include any number of input devices to change any number of operating parameters. In some embodiments, the input device may include a resistance system that may resist movement of the input device. The resistance system may include any type of resistance system, such as a spring, a friction fit, a motor, or other resistance. The resistance system may be used to provide the user a tangible sense of movement when moving the input device. In some embodiments, the input device may include a return mechanism. The return mechanism may include a spring or compliant material that may return the input device to a neutral position. The user may overpower the return mechanism to adjust the input device.

In some embodiments, the exercise equipment includes one or more sensors. In some embodiments, the sensor is configured to detect a motor load. For example, the sensor may be a power consumption sensor, an amperage meter, a current meter, a weight sensor, or a combination thereof. The sensor is configured to detect one or more motor load values, such as, a power, an amperage, a current, a weight, a time, or a combination thereof. The sensor may use power provided by the power supply to operate the sensor, or it may have its own power supply. The sensor is configured to provide the one or more detected values to the exercise manager. In some embodiments, a motor load is the load used to drive the tread belt of the exercise equipment.

In some embodiments, the one or more sensors is configured to detect a point of contact made by each step on the tread deck when an exercise equipment is operated with a first configuration. For example, the one or more sensors may be an optical sensor, electro-mechanical sensors, pneumatic sensors, capacitive sensors, inductive sensors, magnetic sensors, or ultrasonic sensors. These sensors are configured to provide a location and/or the size of area within the tread deck on where a step (e.g., impact) is recorded on. For example, if a location of a step is detected near the sides of the tread deck, or if the size of the step (e.g., impact) is bigger than the estimated size of a feet, it may indicate that the user has lost balance, has experienced a moment of unsteadiness, has tripped, or otherwise lost control of him or herself. In response to detecting that the obtained point of contact is different from the predictive point of contact profile, the exercise equipment may automatically adjust, without any user input, one or more of the operating parameters. For example, the exercise equipment may adjust one or more of the operating parameters to a neutral operating level.

In some embodiments, the exercise manager further includes a storage device. A storage device may store user profile that includes one or more user profile parameters, for example, a user's personal information (e.g., age, weight, height, sex, etc.), and the user's preferences on exercise programs (tensity, length, speed, incline, program, etc.).

In some embodiments, the exercise manager includes a motor load manager configured to create a motor load profile for a user. For example, the motor load manager may analyze the one or more user profile parameters, the one or more exercise equipment settings, the one or more motor load values, the one or more point of contact point information, or a combination thereof to create a motor load profile for the user. For example, a motor load profile may include one or more of an average motor load, a motor load pattern over a period of time, a maximum motor load, a minimum motor load, a point of contact information, or a combination thereof. Examples of a motor load profile are further discussed herein. The motor load profile may be stored on the storage device. In some embodiments, the motor load profile is stored on an external storage device via a network connection.

In some embodiments, the motor load manager is further configured to compare the motor load profile to an obtained motor load values and/or the obtained point of contact information provided by the sensor. For example, by comparing the motor load profile to the obtained motor load values, the exercise manager may detect a change in the motor load. In response to detecting the change in the motor load, the exercise manager may perform a safety action, such as adjusting operation of the exercise equipment (e.g., initiating an emergency stop, or initiating a slowdown), providing a warning (e.g., providing an audible alert sound and/or providing a warning to a user via a display), or a combination thereof. In some embodiments, providing a warning to a user via a display further includes providing a prompt to confirm identity of the individual using the exercise device. For example, a first user may have a different motor load profile than a second user, and hence the second user may trigger a difference on a motor load if operated with the assumption that it is the first user operating it. In some embodiment, the motor load manager may the utilize point of contact information in determining what type of safety action to perform based on the change in the motor load.

In some embodiments, the motor load profile may be based on one or more exercises performed in the past by a user. In some embodiments, the motor load profile may be based on the ongoing (e.g., current) exercise performed by a user. In some embodiments, the motor load profile may be a combination of the past exercise performances and the current exercise performance.

In some embodiments, a motor load manager may receive one or more user profile parameters, one or more exercise equipment settings, one or more historical motor load values, one or more current motor load values, one or more point of contact information, or a combination thereof. The motor load manager may utilize one or more of these to create a motor load profile for a user. For example, a motor load profile may include one or more of an average motor load, a motor load pattern over a period of time, a maximum motor load, a minimum motor load, a point of contact pattern, or a combination thereof.

In some embodiments, a motor load profile includes an average estimated motor load required to operate the exercise machine for a first user with a first set of operating parameters. For example, the motor load manager may use a first user's weight (e.g., user profile parameters), and a first speed of a tread belt to estimate an average motor load for the first user using the exercise equipment with the first speed. The motor load manager may then perform similar analysis with a second speed, third speed, etc., or to a second user, third user, etc.

In some embodiments, a motor load profile may include an estimated minimum (min) and/or maximum (max) motor load required to operate the exercise machine for a first user with a first set of operating parameters. For example, the motor load manager may receive current exercise equipment settings, and current motor load values to estimate min and max motor load required to operate the exercise machine with the current exercise equipment settings.

In some embodiment, the motor load profile includes a motor load pattern over a period of time. For example, motor load manager may use historical motor load values detected by a motor load sensor and stored by a storage device to create a motor load pattern over a period of time. In some embodiments, a motor load pattern may show a change in motor load over a period of time. For example, in treadmill, the motor load value may be higher each time a feet touches the treadmill, compared to times when no feet are touching the treadmill such as when a user is running on a treadmill. In another example, the impact of a feet touching a treadmill when a user is walking may be lower than the impact of a feet touching a treadmill when the user is running. For example, with an exercise bike, the rhythm of pedaling may be different based on the exercise equipment settings used, the fitness of the user, and the strength of both feet (e.g., imbalance on muscle strength and/or injuries on a leg may affect how much workload each pedaling requires). Similarly with a flywheel, the exercise equipment settings, and the fitness of the user may affect how much workload is required.

In some embodiments, a motor load profile additionally includes a point of contact pattern. For example, one or more sensors may detect a point of contact made by each step on the tread deck when an exercise equipment is operated with a first configuration. For example, the one or more sensors may be an optical sensor, electro-mechanical sensors, pneumatic sensors, capacitive sensors, inductive sensors, magnetic sensors, or ultrasonic sensors. In some embodiments, the sensors are configured to provide a point of contact information to the motor load manager. For example, the point of contact information may be a position (e.g., a location) within the tread belt where a step has been detected, and/or the size of area within the tread deck on where a step (e.g., impact) is detected on. Based on the received point of contact information the motor load manager may perform a statistical analysis to determine estimated point of contact, e.g., the estimated position and/or size of the impact on the tread belt which is stored as part of the motor load profile. The estimated point of contact may then be used together with the motor load estimation to determine if the user has lost balance, has experienced a moment of unsteadiness, has tripped, or otherwise lost control of him or herself. For example, if a location of a step is detected near the sides of the tread deck, or if the size of the step (e.g., impact) is bigger than the estimated size of a feet, it may indicate that the user has lost balance, has experienced a moment of unsteadiness, has tripped, or otherwise lost control of him or herself. In response to detecting that the obtained point of contact is different from the predictive point of contact profile, the exercise equipment may automatically adjust, without any user input, one or more of the operating parameters. For example, the exercise equipment may adjust one or more of the operating parameters to a neutral operating level.

In some embodiments, an average current of a motor load profile required to operate a treadmill with six different incline parameters for three different users are provided. The average current with User 3 may be lower than the average current required for Users 1 and 2. Differences on the average current for different users may be explained by the weight of the users, or the speed of the treadmill. For example, User 3 may weigh the least and User 2 may weigh the most of the three. In another example, User 2 may always operate the treadmill on higher speed than Users 1 and 3. For example, User 2 may operate the treadmill on 5 mph speed, User 1 may operate the treadmill on 3 mph, and User 3 may operate the treadmill on 2.5 mph during each incline.

A motor load profile may include minimum, maximum, and average current for a single user based on a speed of a tread belt, in accordance with at least one or more embodiments. As the speed of the tread belt increases, so does the minimum, maximum, and average current required to operate the exercise machine increase. In some embodiments, the increase may or may not be linear.

In some embodiments, a motor load profile with a motor load pattern is based on time, in accordance with at least one or more embodiments. For example, the motor load pattern may be for a treadmill. During t=0 and t=2, the current required to operate a treadmill is 4 amps. At t=3, the current required to operate the treadmill increases to 10 amps. During t=4 and t=6, the current required to operate the treadmill decreases back to 4 amps. At t=7, the current required to operate the treadmill increases again to 10 amps. This same pattern of three consecutive time instances at 4 amps followed by a single time instance at 10 amps may continue along the whole time frame of the motor load profile. It can be concluded that at t=3, t=7, and t=11 is the time frame when a user's feet touches the treadmill, for example, when the user is running and providing heavier weight on the tread belt on those time instances when their feet touches the tread belt.

In some instances, the motor load may be for a flywheel. For example, at t=3, t=7, and t=11 is the time frame when the user pulls the flywheel, and the exercise device provides resistance to the pull. It should be noted that even if the motor load value is a current, any other motor load value could also be used (e.g., a power, an amperage, a current, a resistance, or a combination thereof).

A system may detect a change in the motor load, in accordance with at least one or more embodiments. Over a period of time from t=0 to t=9, the average estimated motor load (e.g., power, amperage, current, resistance, or combination thereof) has been determined to be 4 units. A detected average of motor load has been detected for the same time periods, and the difference between the two has been provided. The closer to 1 the difference is, the more aligned the estimated motor load and the detected motor load are to each other's. As can be seen from FIG. 6, there is not much difference between the two, between t=0 and t=6. However, the exercise equipment has detected significantly decreased motor load values between t=7 and t=9. For example, a significantly decreased motor load values may indicate that the user has fallen off the exercise device or has voluntarily jumped out of it. In some embodiments, a threshold value may be set on how big of a difference is allowed between the estimated average and the detected average to determine that the difference is significant. For example, a difference between 0.9 and 1.1 may be accepted as normal whereas anything below 0.9 or above 1.1 is considered significant. In another example, a difference between 0.8 and 1.2 may be accepted as normal whereas anything below 0.8 or above 1.2 is considered significant.

In response to detecting a significant change on the expected and detected motor load values, the exercise device may perform an action. For example, an action may include adjusting operation of the treadmill to a neutral configuration. In some embodiments, a time threshold may also be set on how quickly an action is performed based on detecting a significant difference. For example, if there is a significant different on two consecutive time periods, the system may perform a safety action. In another example, if there is three or more consecutive time periods, the system may perform a safety action. In yet another example, detecting only one significant difference may be enough to initiate a safety action.

In some embodiments, the estimated value of motor load is based on the motor load profile created previously. The difference between the estimated value and the detected motor load value seems to be non-significant between t=0 and t=7. Between t=8 and t=10, there is significant difference detected, while at t=11 the difference is not significant. As the detected motor load value is significantly higher than expected (i.e., when the user's feet are not touching the tread belt) it can be concluded that the user has most probably fallen on to the belt and is not running anymore, as the detected value between t=8 and t=12 stays around the same (high) value.

In some embodiments, a minimum and maximum values for the motor load may also be used to detect if the detected average or the detected value falls outside of the minimum and maximum values, in which case it is determined that there is a significant change in the motor load value.

They system may use estimated point of contact information to determine that a user has lost balance, has experienced a moment of unsteadiness, has tripped, or otherwise lost control of him or herself while using an exercise equipment. In some embodiments, a motor load profile, includes estimation of point of contact on a tread belt. For example, the estimation may be based on sensor data collected over time, as previously discussed, or it could be automatically set. A danger zone has been established to cover the boarders of the tread belt. Similarly, a safe zone has been provided to cover the rest of the area of the tread belt. In some embodiments, when an impact is detected on a danger zone the system may determine that the user has lost balance, has experienced a moment of unsteadiness, has tripped, or otherwise lost control of him or herself. In some embodiments, when an impact is detected on a danger zone and if a difference is detected on a workload, the system may determine that the user has lost balance, has experienced a moment of unsteadiness, has tripped, or otherwise lost control of him or herself.

The system may use estimated point of contact information to determine that a user has lost balance, has experienced a moment of unsteadiness, has tripped, or otherwise lost control of him or herself while using an exercise equipment. In some embodiments, a motor load profile includes estimation of point of contact on a tread belt. For example, the estimation may be based on sensor data collected over time, as previously discussed, or it could be automatically set. Two separate safe zones have been established based on sensor data collected when the exercise machine is operated with first configuration. In some embodiments, the two separate safe zones may be separately established for each feet. For example, if a left feet is detected on a safe zone that has been established for right feet, and which is outside of the left feet safe zone, it can still be determined to be on a danger zone for the left feet. Similarly, if a right feet is detected outside of right feet safe zone, even if it would be on a left feet safe zone, it will still be determined to be on a danger zone for right feet. For simplicity, the two separate safe zones are here determined to be safe zones for both feet. A danger zone covers the rest of the area of the tread belt. In some embodiments, when an impact is detected on a danger zone the system may determine that the user has lost balanced, has experienced a moment of unsteadiness, has tripped, or otherwise lost control of him or herself. In some embodiments, when an impact is detected on a danger zone and if a difference is detected on a workload, the system may determine that the user has lost balanced, has experienced a moment of unsteadiness, has tripped, or otherwise lost control of him or herself. In response to detecting that the obtained point of contact is different from the predictive point of contact profile, the exercise equipment may automatically adjust, without any user input, one or more of the operating parameters. For example, the exercise equipment may adjust one or more of the operating parameters to a neutral operating level.

A method may include an act of obtaining a motor load. For example, the motor load may be the motor load of an exercise equipment with selected one or more operating parameters.

The method may further include an act of detecting a change in the motor load. For example, detecting the change in the motor load may include comparing a current used to drive the exercise equipment with a selected operating parameters to an estimated current used to drive the exercise equipment with the selected operating parameters and detecting over 10% difference between the two.

The method may further include an act of slowing down the exercise equipment in response to detecting the change in the motor load. For example, slowing down the exercise equipment may be done automatically without user input.

A method may include an act of operating the exercise equipment. For example, operating the exercise equipment with a first configuration.

The method may further include an act of obtaining an obtained motor load. For example, obtaining an obtained motor load of the exercise equipment at the first configuration.

The method may further include an act of comparing the obtained motor load to an estimated motor load. For example, comparing the obtained motor load to an estimated motor load associated with the first configuration.

The method may further include an act of performing (e.g., taking) a safety action. For example, performing a safety action when the obtained motor load is different than the estimated motor load.

A method may include an act of generating one or more predictive motor load profiles. For example, generating one or more predictive motor load profiles for an individual user based on historical motor load information of the treadmill at an operating configuration.

The method may further include an act of detecting a detected motor load pattern. For example, detecting a detected motor load pattern of the treadmill at the operating configuration for the individual user.

The method may further include an act of providing diagnostic information on a state of health of the treadmill. For example, providing diagnostic information on a state of health of the treadmill may be provided based on comparing an obtained motor load patter to a predictive motor load profile. For example, if the obtained motor load used for operating the exercise equipment has increased from the predictive motor load profile, the diagnostic information may suggest one or more maintenance procedures to prevent any further damage and to help extend the service life of the treadmill.

A computer may be used to implement the various computing devices, components, and systems described herein. As used herein, a “computing device” refers to electronic components that perform a set of operations based on a set of programmed instructions. Computing devices include groups of electronic components, client devices, server devices, etc.

In various implementations, the computer system represents one or more of the client devices, server devices, or other computing devices described above. For example, the computer system may refer to various types of network devices capable of accessing data on a network, a cloud computing system, or another system. For instance, a client device may refer to a mobile device such as a mobile telephone, a smartphone, a PDA, a tablet, a laptop, a wearable computing device (e.g., a headset or smartwatch), or exercise device. A client device may also refer to a non-mobile device such as a desktop computer, a server node (e.g., from another cloud computing system), or another non-portable device.

The computer system includes a processing system including a processor. The processor may be a general-purpose single- or multi-chip microprocessor (e.g., an ARM), a special-purpose microprocessor (e.g., a DSP), a microcontroller, a programmable gate array, etc. The processor may be referred to as a CPU. Although the processor is described as a single processor, in an alternative configuration, a combination of processors (e.g., an ARM and DSP) could be used.

The computer system also includes memory in electronic communication with the processor. The memory may be any electronic component capable of storing electronic information. For example, the memory may be embodied as RAM, ROM, magnetic disk storage media, optical storage media, flash memory devices in RAM, on-board memory included with the processor, EPROM, EEPROM, registers, and so forth, including combinations thereof.

The instructions and the data may be stored in the memory. The instructions may be executable by the processor to implement some or all of the functionality disclosed herein. Executing the instructions may involve the use of the data that is stored in the memory. Any of the various examples of modules and components described herein may be implemented, partially or wholly, as instructions stored in memory and executed by the processor. Any of the various examples of data described herein may be among the data that is stored in memory and used during the execution of the instructions by the processor.

A computer system may also include one or more communication interface(s) for communicating with other electronic devices. The one or more communication interface(s) may be based on wired communication technology, wireless communication technology, or both. Some examples of the one or more communication interface(s) include a USB, an Ethernet adapter, a wireless adapter that operates in accordance with an IEEE 1002.11 wireless communication protocol, a Bluetooth® wireless communication adapter, and an IR communication port.

A computer system may also include one or more input device(s) and one or more output device(s). Some examples of the one or more input device(s) include a keyboard, mouse, microphone, remote control device, button, joystick, trackball, touchpad, and light pen. Some examples of the one or more output device(s) include a speaker and a printer. A specific type of output device that is typically included in a computer system is a display device. The display device used with implementations disclosed herein may utilize any suitable image projection technology, such as LCD, LED, gas plasma, electroluminescence, or the like. A display controller may also be provided, for converting data stored in the memory into text, graphics, and/or moving images (as appropriate) shown on the display device.

The various components of the computer system may be coupled together by one or more buses, which may include a power bus, a control signal bus, a status signal bus, a data bus, etc. For clarity, the various buses are described herein as a bus system.

Embodiments of this application may be described according to any of the following sections:

A1. A method for operating an exercise equipment, comprising:
obtaining a motor load of the exercise equipment with selected one or more operating parameters;
detecting a change in the motor load; and
slowing down the exercise equipment in response to detecting the change in the motor load.
   A2. The method of section A1, wherein detecting the change in the motor load further includes detecting decrease or increase in the motor load.
   A3. The method of section A2, wherein detecting decrease in the motor load indicates that a user has fallen off the exercise equipment or has voluntarily jumped out of it.
   A4. The method of section A2, wherein detecting increase in the motor load indicates that a user has fallen on the exercise equipment.
   A5. The method of any of the sections A1-A4, wherein the exercise equipment is a treadmill.
   A6. The method of section A5, wherein the motor load is one or more of amount of energy, amount of power, amount of amperage, or amount of current used to drive a tread belt of the treadmill.
   A7. The method of section A6, wherein obtaining the motor load includes obtaining a current, an amperage, a resistance, a time, a power, or a combination thereof needed to drive the tread belt of the treadmill.
   A8. The method of section A7, further including storing a motor load profile.
   A9. The method of section A8, wherein the motor load profile includes one or more of an average motor load, a motor load pattern over a period of time, a maximum motor load, or a minimum motor load.
   A10. The method of any of the sections A8-A9, wherein the motor load of the motor load profile is an estimated motor load pattern over a period of time used to drive the exercise equipment with a selected operating parameters.
   A11. The method of any of the sections A1-A10, wherein detecting the change in the motor load further includes comparing a detected motor load pattern used to drive the exercise equipment with the selected operating parameters to the estimated motor load pattern used to drive the exercise equipment with the selected operating parameters and detecting over 10% difference.
   A12. The method of any of the sections A9-A11, wherein the motor load of the motor load profile is an estimated current used to drive the exercise equipment with a selected operating parameters.
   A13. The method of section A12, wherein the estimated current used to drive the exercise equipment at the selected operating parameters decreases based on a decrease in operating parameter value.
   A14. The method of section A12, wherein the estimated current used to drive the exercise equipment at the selected operating parameters increases based on an increase in operating parameter value.
   A15. The method of any of the sections A12-A14, wherein detecting the change in the motor load further includes comparing the current used to drive the exercise equipment with the selected operating parameters to the estimated current used to drive the exercise equipment with the selected operating parameters and detecting over 10% difference.
   A16. The method of any of the sections A1-A15, further including automatically providing a warning to a user via a display in response to detecting the change in the motor load.
   A17. The method of any of the sections A1-A16, further including automatically providing an audible alert sound in response to detecting the change in the motor load.
   A18. The method of any of the sections A1-A17, wherein slowing down the exercise equipment further includes automatically slowing down the exercise equipment without user input.
   B1. A method for operating an exercise equipment, comprising:
operating the exercise equipment with a first configuration;
obtaining an obtained motor load of the exercise equipment at the first configuration;
comparing the obtained motor load to an estimated motor load associated with the first configuration; and
performing a safety action when the obtained motor load is different than the estimated motor load.
   B2. The method of section B1, wherein the exercise equipment is a treadmill.
   B3. The method of any of the sections B1-B2, wherein performing the safety action includes adjusting operation of the exercise equipment to a neutral configuration.
   B4. The method of section B3, wherein the neutral configuration includes stopping a tread belt.
   B5. The method of section B4, wherein stopping the tread belt includes initiating an emergency stop.
   B6. The method of section B4, wherein stopping the tread belt includes initiating a slow down to stop the tread belt over a period of time.
   B7. The method of any of the sections B1-B6, wherein the safety action includes providing a warning.
   B8. The method of any of the sections B1-B7, wherein the first configuration includes at least one of a speed of a tread belt or an incline of a tread deck.
   B9. The method of any of the sections B1-B8, further including operating the exercise equipment at a second configuration, wherein the second configuration is different than the first configuration and adjusting the estimated motor load is based on the second configuration.
   B10. The method of any of the sections B1-B9, wherein obtaining the obtained motor load includes obtaining an obtained current used to operate the exercise equipment with the first configuration.
   B11. The method of section B10, further comprising storing a motor load profile, wherein the motor load profile includes the estimated motor load associated with the first configuration.
   B12. The method of section B11, wherein the estimated motor load associated with the first configuration includes an estimated current used to operate the exercise equipment with the first configuration.
   B13. The method of section B12, wherein comparing the obtained motor load to the estimated motor load associated with the first configuration further includes comparing the obtained current to the estimated current.
   B14. The method of section B12, wherein the estimated current used to operate the exercise equipment with the first configuration increases based on increase in user's weight.
   B15. The method of section B12, wherein the estimated current used to operate the exercise equipment with the first configuration increased based on increase in at least one of a speed of a tread belt or in incline of a tread deck.
   C1. A treadmill configured to prevent injuries, comprising:
a motor configured to drive a tread belt of the treadmill;
a sensor configured to measure a detected motor load; and
a processor and memory, the memory including instructions that cause the processor to:
   operate the treadmill with a first configuration;
   receive the detected motor load of the treadmill from the sensor;
   compare the detected motor load to an estimated motor load associated with the first configuration; and
   perform a safety action when the detected motor load is different than the estimated motor load.
      C2. The treadmill of section C1, wherein the safety action includes adjusting operation of the treadmill to a neutral configuration.
      C3. The treadmill of section C2, wherein the neutral configuration includes stopping the tread belt.
      C4. The treadmill of section C3, wherein stopping the tread belt includes initiating an emergency stop.
      C5. The treadmill of section C3, wherein stopping the tread belt includes initiating a slow down to stop the tread belt over a period of time.
      C6. The treadmill of any of the sections C1-C5, wherein the safety action includes providing a warning.
      C7. The treadmill of any of the sections C1-C6, wherein the first configuration includes at least one of a speed of the tread belt or an incline of a tread deck.
      C8. The treadmill of any of the sections C1-C7, further including operating the treadmill at a second configuration, wherein the second configuration is different than the first configuration and adjusting the estimated motor load based on the second configuration.
      C9. The treadmill of any of the sections C1-C8, wherein detecting the detected motor load includes detecting a detected current used to operate the treadmill with the first configuration.
      C10. The treadmill of section C9, further comprising storing motor load profile wherein the motor load profile includes the estimated motor load associated with the first configuration.
      C11. The treadmill of section C10, wherein the estimated motor load associate with the first configuration includes an estimated current used to operate the treadmill with the first configuration.
      C12. The treadmill of section C11, wherein comparing the detected motor load to the estimated motor load associated with the first configuration further includes comparing the detected current to the estimated current.
      C13. The treadmill of section C11, wherein the estimated current used to operate the treadmill with the first configuration increases based on increase in user's weight.
      C14. The treadmill of section C11, wherein the estimated current used to operate the treadmill with the first configuration increased based on increase in at least one of a speed of the tread belt or an incline of a tread deck.
      C15. The treadmill of any of the sections C1-C14, wherein the sensor configured to measure the detected motor load includes detecting a current used to operating the treadmill with the first configuration.
      C16. The treadmill of any of the sections C6-C15, wherein providing the warning further including a speaker configured to provide an audible alert sound.
      C17. The treadmill of any of the sections C1-C16, wherein the sensor is a power consumption sensor, an amperage meter, a current meter, a weight sensor, a position sensor, or a combination thereof.
      D1. A method for operating a treadmill comprising:
detecting a detected motor load pattern of the treadmill at a first configuration over a first period of time;
generating a predictive motor load profile based on the detected motor load pattern of the treadmill at the first configuration;
detecting a change in the detected motor load pattern compared to the predictive motor load profile; and
performing an action on the treadmill in response to detecting the change in the detected motor load pattern.
   D2. The method of section D1, wherein detecting the change in the detected motor load pattern includes detecting an increase or decrease in the detected motor load pattern.
   D3. The method of any of the sections D1-D2, wherein the predictive motor load profile is generated for an individual user.
   D4. The method of any of the sections D1-D3, wherein the detected motor load pattern includes at least one or more of a rhythm of steps, a force applied by each step, or a point of contact made by each step to a tread deck.
   D5. The method of any of the sections D1-D4, wherein performing the action includes adjusting operation of the treadmill to a neutral configuration.
   D6. The method of section D5, wherein the neutral configuration includes stopping a tread belt.
   D7. The method of section D6, wherein stopping the tread belt includes initiating an emergency stop.
   D8. The method of section D6, wherein stopping the tread belt includes initiating a slow down to stop the tread belt over a second period of time.
   D9. The method of any of the sections D1-D8, wherein performing the action includes providing a warning.
   D10. The method of section D9, wherein providing the warning further includes providing a prompt to confirm identity of an individual using of the treadmill.
   D11. The method of section D9, wherein providing the warning further includes providing an alert sound.
   D12. The method of any of the sections D8-D11, wherein initiating the slow down to stop the tread belt over a period of time further includes gradually reducing a power provided to a motor.
   D13. The method of section D7, wherein initiating the emergency stop further includes disabling power provided to a motor.
   E1. A method for operating a treadmill, comprising:
generating one or more predictive motor load profiles for an individual user based on historical motor load information of the treadmill at an operating configuration;
detecting a detected motor load pattern of the treadmill at the operating configuration for the individual user; and
providing diagnostic information on a state of health of the treadmill in response to detected that the detected motor load pattern is different than the one or more predictive motor load profiles.
   E2. The method of section E1, wherein the diagnostic information may suggest one or more maintenance procedures to prevent damage, help extend a service life of the treadmill, or a combination thereof.

Embodiments of the present disclosure may thus utilize a special purpose or general-purpose computing system including computer hardware, such as, for example, one or more processors and system memory. Embodiments within the scope of the present disclosure also include physical and other computer-readable media for carrying or storing computer-executable instructions and/or data structures, including applications, tables, data, libraries, or other modules used to execute particular functions or direct selection or execution of other modules. Such computer-readable media can be any available media that can be accessed by a general purpose or special purpose computer system. Computer-readable media that store computer-executable instructions (or software instructions) are physical storage media. Computer-readable media that carry computer-executable instructions are transmission media. Thus, by way of example, and not limitation, embodiments of the present disclosure can include at least two distinctly different kinds of computer-readable media, namely physical storage media or transmission media. Combinations of physical storage media and transmission media should also be included within the scope of computer-readable media.

Both physical storage media and transmission media may be used temporarily store or carry, software instructions in the form of computer readable program code that allows performance of embodiments of the present disclosure. Physical storage media may further be used to persistently or permanently store such software instructions. Examples of physical storage media include physical memory (e.g., RAM, ROM, EPROM, EEPROM, etc.), optical disk storage (e.g., CD, DVD, HDDVD, Blu-ray, etc.), storage devices (e.g., magnetic disk storage, tape storage, diskette, etc.), flash or other solid-state storage or memory, or any other non-transmission medium which can be used to store program code in the form of computer-executable instructions or data structures and which can be accessed by a general purpose or special purpose computer, whether such program code is stored as or in software, hardware, firmware, or combinations thereof.

A “network” or “communications network” may generally be defined as one or more data links that enable the transport of electronic data between computer systems and/or modules, engines, and/or other electronic devices. When information is transferred or provided over a communication network or another communications connection (either hardwired, wireless, or a combination of hardwired or wireless) to a computing device, the computing device properly views the connection as a transmission medium. Transmission media can include a communication network and/or data links, carrier waves, wireless signals, and the like, which can be used to carry desired program or template code means or instructions in the form of computer-executable instructions or data structures and which can be accessed by a general purpose or special purpose computer.

Further, upon reaching various computer system components, program code in the form of computer-executable instructions or data structures can be transferred automatically or manually from transmission media to physical storage media (or vice versa). For example, computer-executable instructions or data structures received over a network or data link can be buffered in memory (e.g., RAM) within a network interface module (NIC), and then eventually transferred to computer system RAM and/or to less volatile physical storage media at a computer system. Thus, it should be understood that physical storage media can be included in computer system components that also (or even primarily) utilize transmission media.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

1. A method for operating an exercise equipment, comprising:
obtaining a motor load of the exercise equipment with selected one or more operating parameters;
detecting a change in the motor load; and
slowing down the exercise equipment in response to detecting the change in the motor load.

2. The method of claim 1, wherein the exercise equipment is a treadmill.

3. The method of claim 2, wherein the motor load is one or more of amount of energy, amount of power, amount of amperage, or amount of current used to drive a tread belt of the treadmill.

4. The method of claim 3, wherein obtaining the motor load comprises obtaining a current, an amperage, a resistance, a time, a power, or a combination thereof needed to drive the tread belt of the treadmill.

5. The method of claim 4, further comprising storing a motor load profile.

6. The method of claim 5, wherein the motor load profile comprises one or more of an average motor load, a motor load pattern over a period of time, a maximum motor load, or a minimum motor load.

7. The method of claim 6, wherein the motor load of the motor load profile is an estimated motor load pattern over a period of time used to drive the exercise equipment with a selected operating parameters.

8. The method of claim 6, wherein the motor load of the motor load profile is an estimated current used to drive the exercise equipment with a selected operating parameters.

9. A method for operating an exercise equipment, comprising:
operating the exercise equipment with a first configuration;
obtaining an obtained motor load of the exercise equipment at the first configuration;
comparing the obtained motor load to an estimated motor load associated with the first configuration; and
performing a safety action when the obtained motor load is different than the estimated motor load.

10. The method of claim 9, wherein performing the safety action comprises adjusting operation of the exercise equipment to a neutral configuration, wherein the neutral configuration comprises stopping a tread belt.

11. The method of claim 10, wherein stopping the tread belt comprises initiating an emergency stop.

12. The method of claim 10, wherein stopping the tread belt comprises initiating a slow down to stop the tread belt over a period of time.

13. The method of claim 9, wherein obtaining the obtained motor load comprises obtaining an obtained current used to operate the exercise equipment with the first configuration.

14. The method of claim 13, further comprising storing a motor load profile, wherein the motor load profile comprises the estimated motor load associated with the first configuration.

15. The method of claim 14, wherein the estimated motor load associated with the first configuration comprises an estimated current used to operate the exercise equipment with the first configuration.

16. The method of claim 15, wherein comparing the obtained motor load to the estimated motor load associated with the first configuration further comprises comparing the obtained current to the estimated current.

17. A treadmill configured to prevent injuries, comprising:
a motor configured to drive a tread belt of the treadmill;
a sensor configured to measure a detected motor load; and
one or more processors and one or more memories, the one or more memories storing instructions that cause the one or more processors to:
operate the treadmill with a first configuration;
receive the detected motor load of the treadmill from the sensor;
compare the detected motor load to an estimated motor load associated with the first configuration; and
perform a safety action when the detected motor load is different than the estimated motor load.

18. The treadmill of claim 17, wherein the safety action comprises providing a warning.

19. The treadmill of claim 18, wherein providing the warning further comprising a speaker configured to provide an audible alert sound.

20. The treadmill of claim 17, wherein the sensor is a power consumption sensor, an amperage meter, a current meter, a weight sensor, a position sensor, or a combination thereof.
